(19) United States
(12) Patent Application Publication
(10) Pub. No.: US 2024/0016397 A1
(43) Pub. Date: Jan. 18, 2024

(54) ESTIMATION OF BLOOD PRESSURE USING BALLISTOCARDIOGRAM AND PERIPHERAL PHOTOPLETHYSMOGRAM

(71) Applicants: Hill-Rom Services, Inc., Batesville, IN (US); Georgia Tech Research Corp., Atlanta, GA (US)

(72) Inventors: Hewon Jung, Atlanta, GA (US); David J. Lin, Atlanta, GA (US); Jacob P. Kimball, Douglasville, GA (US); Omer T. Inan, Marietta, GA (US); Eric D. Agdeppa, Cary, NC (US); Timothy J. Receveur, Guilford, IN (US)

(21) Appl. No.: 18/352,468

(22) Filed: Jul. 14, 2023

Related U.S. Application Data
(60) Provisional application No. 63/389,340, filed on Jul. 14, 2022.

(51) Int. Cl. A61B 5/021; A61B 5/024

(52) U.S. Cl. CPC A61B 5/02108; A61B 5/02416

(57) ABSTRACT

Algorithms for continuous BP monitoring using the load cell ballistocardiogram and the finger/toe photoplethysmogram (PPG) signals. This disclosure includes two different approaches; (1) a conventional pulse transit time-based model and (2) a U-Net-based model to predict BP from ballistocardiogram and PPG signals. In pulse transit time-based models, the pulse transit time was acquired through signal processing and linear regression was performed on its inverse to estimate BP. In the U-Net-based model, the source signals (ballistocardiogram and PPG) were translated to BP waveforms from which the BP values were estimated after calibration.

### CROSS-REFERENCE TO RELATED APPLICATIONS

This application claims priority under 35 U.S.C. § 119(e) to U.S. Provisional Patent Application No. 63/389,340, filed Jul. 14, 2022, which is expressly incorporated by reference herein.

### BACKGROUND

Monitoring vital signs is one of the most common and essential clinical interventions to assess patients physiologic states and apply adequate treatment promptly. In most cases, critical inpatient events are known to be preceded by warning signs for about 8-24 hours that usually appear as an abnormal range in measured vitals. Early recognition of such signs and thus the patient deterioration in hospitals is crucial to prevent serious adverse events leading to unplanned admission to the intensive care unit (ICU) or prolonged hospitalization, long-term disability, or even unexpected deaths.

To quantify the deterioration in physiologic state, the early warning score systems have been widely used in hospitals. For example, the modified early warning score scores a risk of adverse events from a set of vitals including heart rate (HR), respiration rate (RR), blood pressure (BP), oxygen saturation (SpO2) and body temperature. Though the early warning score has shown success in detecting medical emergencies and improving patient outcomes, one of the limitations is the intermittency in vital measurement. In general hospital wards, vitals are usually measured with conventional monitoring devices. These devices are often costly and cumbersome to use, limiting vital measurements to two to three times a day. Therefore, the early warning system is also limited by this intermittent nature thus leading to failure in recognizing patient deterioration especially in low-equipped settings or at night when patients are less intensively monitored.

To mitigate this problem, continuous vitals monitoring solutions at the bedside have been proposed to allow early detection of deteriorating trends in vitals. Recent studies have shown that continuous monitoring in combination with automated alerts improves patient outcomes. In particular, hospital bed systems using force sensors such as the load cell and piezoelectric film have shown robust, continuous measurements of HR and RR. Along with the HR and RR, BP is also an important measurement to provide a complete set of vitals required to compute an early warning score and allow accurate clinical assessment.

One conventional approach to non-invasive continuous BP monitoring is through pulse transit time (PTT) or pulse arrival time (PAT). The pulse transit time is defined as the time elapsed for the pulse wave to propagate along the length of the arterial tree and is known to be inversely correlated to the BP. To acquire non-invasive pulse transit time, two timing references, one proximal and the other distal to the heart are required. In practice, a combination of signals such as the electrocardiogram (ECG), photoplethysmogram (PPG) measured from an extremity location such as a fingertip or toe, and cardiogenic vibration signals such as the ballistocardiogram or seismocardiogram could be used as the timing references. In particular, the pulse transit time is often acquired as the timing delay between the fiducial points in ballistocardiogram (i.e. I, J, and K locations shown in FIG. 3) or seismocardiogram and the PPG. The pulse transit time has been found to be highly correlated to the BP in both the controlled lab environment and at-home settings. Similarly, the pulse arrival time is defined as the timing delay between the ECG R-peak and the foot of the PPG. Along with pulse transit time, the pulse arrival time has been widely used to monitor BP due to its convenience in measurement. However, the pulse arrival time has been demonstrated to be less correlated to BP compared to pulse transit time as it includes the pre-ejection period (PEP) which is determined by ventricular properties and autonomic state rather than just BP.

Besides using pulse transit time or pulse arrival time to estimate BP, some studies estimated BP using multiple timing-related or morphological features from the ballistocardiogram and PPG signals as inputs to the machine learning (ML) model. For example, pulse transit time or pulse arrival time features extracted from various locations, the PPG morphological features and some physiological features including HR were used to estimate BP through ML models such as multiple linear regression (MLR), support vector regression (SVR) and tree-based models. Some studies also demonstrated BP estimation using ballistocardiogram signals alone. Features from multi-channel ballistocardiogram recordings have been used to relatively track BP values and a bidirectional long short-term memory network (bi-LSTM) to estimate systolic blood pressure (SBP) and diastolic blood pressure (DBP) from a two-channel ballistocardiogram system. Also, some studies used convolutional neural network (CNN), LSTM, or a combination of two to predict SBP and DBP from the ECG and PPG.

Recently, end-to-end approaches for estimating the BP through translation of PPG signals to BP waveforms have been widely studied. For example, variants of the original U-Net model have been proposed. With the recent success of generative adversarial network (GAN) models in generating realistic images or speech signals, GAN models have also been applied to 1D biosignals as well. For example, the use of cycle GAN models to generate model realistic BP waveforms from the PPG signals alone have been used. Such end-to-end approaches have suggested either a globalized model or the models requiring less frequent calibration; however, most of aforementioned studies were done on the publicly available Multiparameter Intelligent Monitoring in Intensive Care II (MIMIC-II) Wave-form dataset. A need to develop and confirm a generalized approach.

### SUMMARY

The present disclosure includes one or more of the features recited in the appended claims and/or the following features which, alone or in any combination, may comprise patentable subject matter.

According to the present disclosure, a method of estimating a blood pressure of a person comprises collecting a signal of each of a plurality of load cells supporting the person to determine a ballistocardiogram, collecting a signal from a photoplethysmogram signal from an appendage of the person, and applying a model to the signals to determine an estimate of the blood pressure of the person.

In some embodiments, the model includes processing the ballistocardiogram and photoplethysmogram signal to filter the signals, determining a timing delay between the fiducial points in the ballistocardiogram signal and the photoplethysmogram signal to determine a pulse transit time, and estimating the blood pressure of the person by calculating the inverse of the pulse transit time.

In some embodiments, the model includes applying a deep learning model to infer the person's blood pressure. The deep learning model may include a contractive path and an expansive path. The contractive path may reduce the dimension of signal by half while doubling the number of channels for each layer. The contractive path may include cascaded layers of convolution, batch normalization, and non-linear activation. The expansive path may include de-convolutions.

In some embodiments, the method further comprises calibrating the model for the specific person. In some embodiments, the calibration is for the absolute estimation of BP from pulse transit time for the specific person. In some embodiments, the calibration accounts for the specific posture of the person as the model is being applied.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a diagrammatic representation of the person supported on a patient support apparatus embodied as a hospital bed, the hospital bed having four load cells supporting the weight of the person;

FIG. 2 is a diagrammatic representation of sensor signals of the present disclosure being fed to and analog-digital converter;

FIG. 3 is a diagrammatic representation of a protocol used to induce variations in an the actual blood pressure of a person supported on the patient support apparatus of FIG. 1;

FIG. 4 is normalized plot of the response measured during a single heart-beat cycle using four different measurement approaches;

FIG. 5 is a histogram of the distribution of a systolic blood pressure of a first training group of individuals according to the present disclosure;

FIG. 6 is a histogram of the distribution of a diastolic blood pressure of the first training group of individuals;

FIG. 7 is a histogram of the distribution of a systolic blood pressure of a second training group of individuals according to the present disclosure;

FIG. 8 is a histogram of the distribution of a diastolic blood pressure of the second training group of individuals;

FIG. 9 is an overview of the signal processing employed using two different models to perform signal processing to establish a blood pressure value for a given person supported on the patient support apparatus of FIG. 1;

FIG. 10 is a diagrammatic representation of a U-Net architecture employed to perform signal processing according to one aspect of the present disclosure;

FIG. 11 is a table presenting the analysis of the estimation error exhibited by the models of the present disclosure;

FIG. 12 is a correlation plot of the estimated measurement of systolic blood pressure of several persons as measured in different postures correlated to the actual systolic blood pressure;

FIG. 13 is a correlation plot of the estimated measurement of diastolic blood pressure of several persons as measured in different postures correlated to the actual diastolic blood pressure;

FIG. 14 is a correlation plot of the estimated measurement of mean arterial blood pressure of several persons as measured in different postures correlated to the actual mean arterial blood pressure;

FIG. 15 is a plot of the error distribution of the measured systolic blood pressure of several persons as measured in different postures correlated to the actual systolic blood pressure;

FIG. 16 is a plot of the error distribution of the measured diastolic blood pressure of several persons as measured in different postures correlated to the actual diastolic blood pressure;

FIG. 17 is a plot of the error distribution of the measured mean arterial blood pressure of several persons as measured in different postures correlated to the actual mean arterial blood pressure; and

FIG. 18 is a table providing a summary of the calculated estimation error for select persons as determined using the approaches of the present disclosure.

### DETAILED DESCRIPTION

According to the present disclosure, blood pressure (BP) is estimated using a photoplethysmogram (PPG) and the load-cell ballistocardiogram signals in two different approaches: the conventional pulse transit time-based model and a deep learning (DL)-based model. The disclosed algorithms of the model were empirically evaluated on a dataset collected from 20 healthy subjects with recordings consisting of ECG, finger PPG, toe PPG, 4-channel load cell ballistocardiogram and the continuous BP from the finger BP cuff measured in four different postures on a hospital bed 10. The performance of the model was established from several angles. The effects of different source signals: finger PPG, and toe PPG in the pulse transit time analysis, and comparison between multi-modal and single-modal models in DL methods were determined. Also, performance was established under different calibration schemes to understand the effects of postural variability in the test subject. Finally, the performances between the two different approaches were compared to explore the advantages and limitations of each method. In the analysis, twenty healthy subjects (Male: 11, Female: 9, Age: 24.3±3.3; Weight: 67.29±14.4 kg; Height: 171.2±10.3 cm) who had not been diagnosed with cardiovascular or respiratory diseases were recruited for the human subjects study.

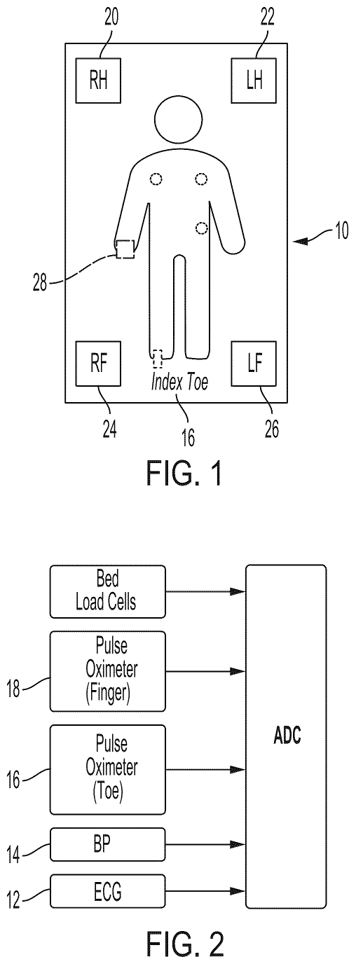

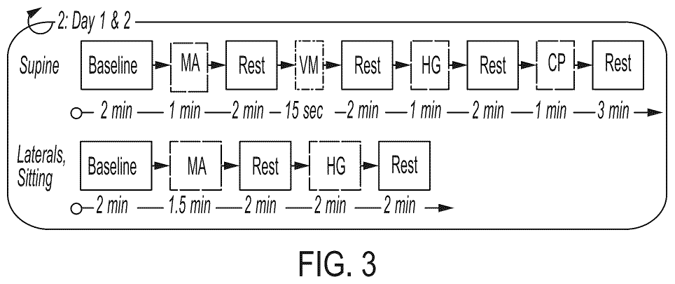

FIG. 1 shows the test setup used for the analysis. Each subject laid down on the patient bed 10 (a Centrella®bed from Hill-Rom) in four different postures with the electrocardiogram (ECG), photoplethysmogram (PPG), and with continuous finger cuff blood pressure (BP) sensor attached to their body. In each posture, a series of perturbations followed by rest periods were performed to modulate BP. Referring to FIG. 3, a mental arithmetic (MA) exercise was conducted in which subjects were given a three-digit integer and were told to add the sum of the digits to the number repeatedly for 1 minute. Then, a Valsalva maneuver (VM) was performed to induce a transient decrease in BP, in which subjects held their breath for fifteen (15) seconds and attempted to forcefully breathe out as if blowing up a balloon while closing their mouth and pinching their nose. In a hand-grip exercise (HG), subjects squeezed an adjustable resistance hand gripper for up to 2 minutes. Finally, during a cold pressor (CP) process, subjects immersed their left hand without sensors in a bucket of ice water for 1 minute to increase BP.

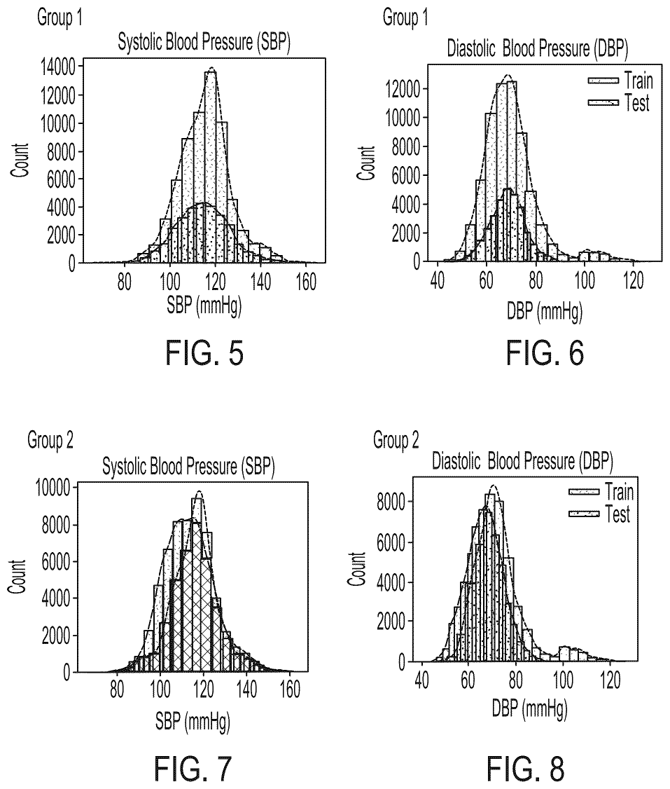

As part of the test protocol, perturbations were introduced to induce large variability in BP such that the model or the calibration curve could be trained and tested on the wide range of BP values. In addition, these perturbations were easily done in static positions to reduce the effect of motion artifacts on the recorded signals. FIGS. 5-8 illustrate histograms comparing the train and test sets for various distributions of BP values. FIG. 5 shows the values for systolic (SBP) of a first group of test subjects and FIG. 7 shows the same values for a second group of test subjects. FIG. 6 shows the test and train values of diastolic pressure (DBP) for the first group and FIG. 8 shows the same values for the second group.

During the protocol, the perturbations were repeated for four different postures, supine, left/right lateral, and sitting, to investigate the effects of postural variability. The Valsalva maneuver and cold pressor were conducted only for the supine posture to reduce the overall protocol length. For the sitting posture, the bed was articulated such that the head-of-bed angle reached 45°. Among the twenty subjects recruited, seventeen subjects visited the lab again within a month for follow-up measurements to investigate the day-to-day variability. The protocol for the follow-up visit was the same as the first visit. In the evaluation of the model, only the subjects who completed the full protocol were included and one subject was excluded due to noisy reference blood pressure recording.

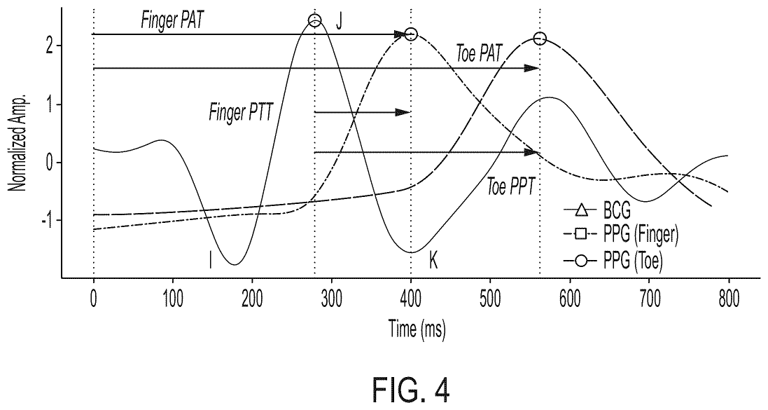

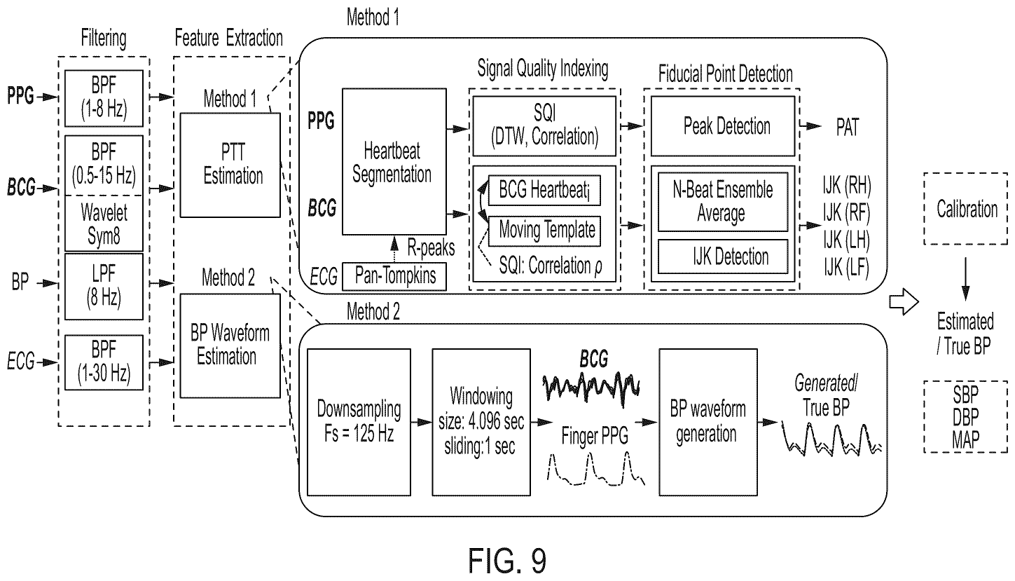

The protocol was performed with ECG, ballistocardiogram, and the ground truth continuous BP being recorded. The placement of all the sensors signal is shown in FIG. 1. An example plot for a typical PPG and ballistocardiogram for one heartbeat cycle is shown in FIG. 4. For the ECG signal, adhesive Ag/AgCl electrodes 12 were placed in lead II configuration. The ECG signals were amplified and acquired through a wireless module (BN-EL50 from Biopac Systems). Ballistocardiogram signals were acquired from the four load cells 20, 22, 24, 26 embedded on the hospital bed 10. The outputs from the load cells 20, 22, 24, 26 were amplified through a custom-designed analog front end to obtain ballistocardiogram signals as disclosed in H. Jung, J. et al., “Accurate Ballistocardiogram Based Heart Rate Estimation Using an Array of Load Cells in a Hospital Bed,” IEEE Journal of Biomedical and Health Informatics, 2021, which is incorporated by reference herein. Also, PPG signals were measured from the right index finger and toe using a pulse oximeter sensor 16 (Model OXY100E by Biopac Systems). To obtain the ground truth BP values, a finger-cuff BP sensor 14 (Model ccNexfin by Edwards Lifesciences) based on the volume-clamp methodology was placed on the same hand (right) as the finger pulse oximeter 18, acquiring a reference measurement of continuous beat-by-beat BP. From each BP beat, SBP, DBP, and mean arterial BP (MAP) were derived as maximum, minimum, and addition of two-thirds of SBP and one-third of DBP. To ensure the calibration of the finger-cuff BP sensor 14 was done properly, at the beginning of each posture, the BP measurements were also taken from an oscillometric BP cuff (not shown) (From Omron, Kyoto, Japan). Recordings for each posture only started when the BP values from the oscillometric cuff and the finger cuff 18 closely agreed with each other. All signals were recorded through an MP160 data acquisition system (Model DAQ by Biopac Systems) at a sampling rate of 1 kHz. FIG. 9 depicts the overall signal processing pipeline. All signal processing and statistical analyses were performed using Python 3.7. Before extracting features, ECG, ballistocardiogram, and PPG signals were first band-pass filtered using Butterworth infinite impulse response (IIR) filters. The cut-off frequencies were 1-8 Hz for PPG, 0.5-15 Hz for ballistocardiogram, and 1-30 Hz for ECG to remove high-frequency noise out of cardiac frequency range and baseline wander caused by respiration. In addition, the continuous BP waveform was low-pass filtered with an IIR filter at the cut-off of 3.5 Hz to smooth out the waveform while maintaining the DC values. To offset the non-linear phase response of the IIR filter, filtering was done in both the forward and reverse directions.

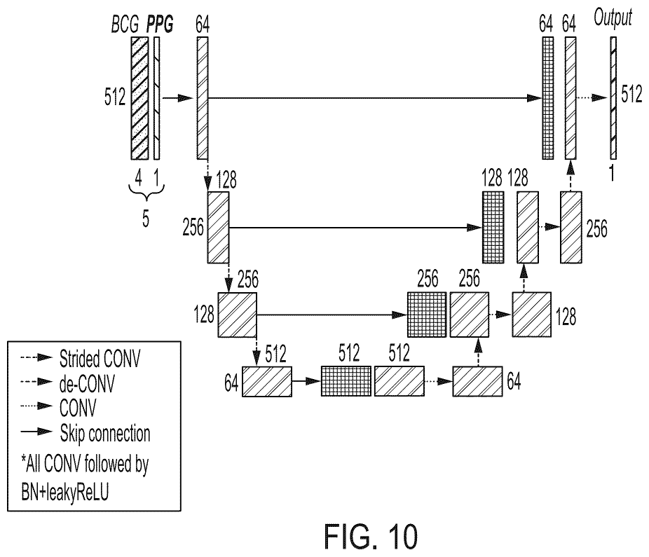

After filtering, two different approaches were employed for the extraction of features relevant to BP from ballistocardiogram and PPG signals. In the first, the signal processing-based extraction of pulse transit time was used to investigate the estimation of BP by leveraging the physiologically known relationship (i.e. the inverse linear correlation) between pulse transit time and the BP was performed. The second approach uses the U-Net (as disclosed in O. Ronneberger, P. Fischer, and T. Brox, “U-net: Convolutional networks for biomedical image segmentation,” in Lecture Notes in Computer Science (including subseries Lecture Notes in Artificial Intelligence and Lecture Notes in Bioinformatics), vol. 9351, 2015), incorporated by reference herein; a DL architecture that was originally proposed for the segmentation of medical images and has shown a promise in many applications including signal to signal translation for cardiac waveforms and denoising of time-series signals. The BP waveform was generated with the U-Net architecture from the 4-channel ballistocardiogram and finger PPG signals to estimate the BP using the architecture shown in FIG. 10.

Before extracting features, the R-peaks of ECG were first detected using the Pan-Tompkins algorithm disclosed in J. Pan and W. J. Tompkins, “A Real-Time QRS Detection Algorithm,” IEEE Transactions on Biomedical Engineering, vol. BME-32, no. 3, 1985, which is incorporated by reference herein. Detected R-peaks were then used to segment PPG, ballistocardiogram, and BP waveforms into cardiac cycles. Then, the signal quality of these heartbeat-indexed signals was assessed for each subject using the signal quality assessment disclosed in Q. Li and G. D. Clifford, “Dynamic time warping and machine learning for signal quality assessment of pulsatile signals,” Physiological Measurement, vol. 33, no. 9, 2012, (“Li”), which is incorporated by reference herein, to remove the segments corrupted by the motion artifacts.

For PPG signals, the signal quality index (SQI) was computed for each heartbeat as described in Li. The method quantifies the similarity between each heart-beat and the template by four different methods including the direct correlation, dynamic time warping (DTW), linear resampling, and clipping detection. Heartbeats that were possibly corrupted were removed using the threshold suggested in Li. A similar approach was taken for ballistocardiogram signals in which the correlation between each heartbeat and the template was computed. Instead of thresholding on the computed SQIs, heartbeats with SQI that belong to the bottom 20 percent were removed. For both PPG and ballistocardiogram, the template was generated for each heartbeat cycle from the moving average of the last 30 beats. All channels, including the finger and toe for PPG and four load cell channels for ballistocardiogram, were processed in the same way.

Because the finger BP cuff sensor 14 intermittently performs an automatic pressure recalibration step, BP heartbeats during this period are generally characterized by a small physiological implausible variability within one cardiac cycle. Affected beats were removed using the correlation SQI and thresholding on the standard deviation. After removing low-fidelity heartbeats through the aforementioned signal quality assessment process, each heartbeat-indexed signal was ensemble-averaged using 8-beat sliding windows to acquire more robust heartbeats for the fiducial point detection. The detection of timing references for pulse transit time computation—the foot/peak of the PPG and the IJK complex of ballistocardiogram described in FIG. 1 was done similarly to the previous studies. In particular, the locations for the maximum 1st order derivative was detected.

For the robust detection of I, J, and K-waves in ballistocardiogram heartbeats, a sliding template matching algorithm was used, in which extrema were found in each heartbeat using fiducial points in the template as references. For the initial few beats in the recordings, a physiologically realistic constraint for possible J-wave location (i.e. within 120-350 ms from ECG R-peak) was imposed to ensure robust I, J, and K detection on initial templates. Subsequent templates for each heart beat were formed using the previous 30 beats.

Finally, the pulse transit time was calculated as the difference of the proximal timing reference, the J-wave of the corresponding ballistocardiogram heartbeat, and distal timing reference, location of the maximum 1st order derivative on the finger or toe PPG. Unlike the previous studies where the pulse transit time was commonly computed as the timing delay between the I-wave of ballistocardiogram and foot of the PPG, J-wave of ballistocardiogram and maximum 1st order derivative of the PPG were used in to ensure that the same morphological peak was consistently chosen across subjects and postures. Occasionally, the I-wave in ballistocardiogram is challenging to detect, especially when the subjects are in lateral postures. To avoid inconsistency in computed pulse transit time arising from I-wave detection error, the J-wave, which is relatively less challenging to find was utilized. As the J-wave comes after the I-wave, to ensure that the J-wave occurs before the distal timing point in PPG, the maximum 1st order derivative was used instead of the foot. These steps were repeated for each ballistocardiogram channel.

Though the conventional pulse transit time-based method has advantages in that it is physiologically interpretable, requires less training data, and has shown great success in many studies, it requires multiple signal processing steps that are often prone to error. For example, the pulse transit time-based method would require detection of heartbeats in ballistocardiogram and PPG signals assuming the reference ECG signal is not available, signal quality assessment, detection of fiducial points, and channel selection/combination. Especially, detection of I, J, and K-waves in ballistocardiogram could get challenging in lateral postures. Though errors in each signal processing step could be minimized by leveraging some advanced techniques, all steps together could lead to a great level of error, leading to inaccurate pulse transit time extraction. A DL-based approach provides a more end-to-end approach that replaces the error-prone signal processing steps with the convolutional neural networks. Therefore, in the second approach for the feature extraction, U-Net DL architecture was used.

In U-Net DL approach, the finger PPG and 4-channel ballistocardiogram signals were used as inputs (i.e. source signals) and the BP waveforms measured by the finger BP cuff were used as the outputs (i.e. target signal). Finger PPG was used instead of using toe PPG or both PPG sources to match the signals in external dataset used for the model training as described in the later section.

For each subject and posture, the time-series PPG, ballistocardiogram, and the BP recordings were first filtered and then downsampled to 125 Hz. Note that the downsampled signals were used to accelerate model training and 125 Hz was chosen. Both the source and target signals were then segmented into 512-samples long (4.096 seconds) windows with 125 samples (1 second) overlap between the windows and each window served as a single training/testing instance for the model.

To remove the windows with the invalid BP waveforms (i.e. the BP signals recorded while the finger BP sensor was calibrating), the windows containing the invalid BP beats identified in the process described in Section II-C-(1) were rejected. To generate BP waveforms from PPG and ballistocardiogram signals, the U-Net architecture was used. The overall architecture is composed of two parts—the contractive and expansive path— as shown in FIG. 10.

The contractive path implementation is similar to the original U-Net model which consists of cascaded layers of convolution, batch normalization and non-linear activation. The contractive path takes the 4-channel ballistocardiogram signals and a single channel PPG signal stacked along the channel dimension as an input. The input has a dimension of Nmini—batch 5 512, where 512 represents the signal length from aforementioned windowing scheme. Nmini—batch size was set as 40.

The contractive path reduces the dimension of the input by half while doubling the number of channels for each layer. Instead of using the max-pooling layer in the original U-Net, to reduce the dimension of the signal, a strided convolution (strided CONV) with stride of 2 and the kernel size of 11 were used in this work. This was followed by a batch normalization (BN) to accelerate the model training and leaky ReLU (Rectified Linear Unit) as a non-linear activation. These alterations were chosen based on the previous signal to signal translation problems using U-Net inspired DL architectures. The contractive path allows the model to learn a compact representation of the input data by recursively reducing the dimension while increasing the number of channels.

The expansive path mirrors the structure in the contractive path to restore the original signal dimension through de-convolutions (i.e. transposed convolution). Each layer first performs transposed convolution on the output from the previous layer. It also takes the feature map from the corresponding layer in the contractive path through the skip connections and concatenates with the output from the transposed convolution along the channel dimension. Then, convolution is performed on the concatenated feature map to halve the number of channels. Concatenating high resolution features from the contracting path allows the model to better construct target signals during the expansive path. For the final layers, instead the halving the number of channels, the channel dimension was reduced to 1 to output the final single-channel BP waveform. As done in the contractive path, the transposed convolution was followed by the BN and the leaky ReLU. For the model training, in addition to the dataset collected from the protocol, a publicly available dataset (referred to as “public BCG dataset” in this work) was also used to increase the number of training instances. In the public BCG dataset, the ECG, PPG, 4-channel load cell ballistocardiogram, and continuous BP waveform from the finger BP cuff were recorded at a sampling rate of 1 kHz. The signals were downsampled to 125 Hz before feeding into the model. In total, the data were collected from 40 subjects (17 males, 23 females) while staying motionless on the bed. Unlike the in-house dataset collected subjects were not performing any tasks to modulate the BP nor were they asked to vary postures.

For the training, 30 subjects from the public BCG dataset were included along with 10 subjects from the in-house dataset. The remaining 10 subjects in the public BCG dataset were included in the validation set. Of the remaining subjects in the in-house dataset, 3 were used for validation and 5 were used for testing. The model training was performed twice with the same model architecture and hyperparameters but with different training, validation, and test split. A different set of training/validation/test subjects were chosen from the in-house dataset to evaluate the performance on a sufficient number of subjects from the in-house dataset. The two different splits have led to 65547/13933/20770 and 54935/17253/28062 instances for training, validation, and testing respectively. The distribution for each scheme is shown in FIGS. 5-8 as discussed above.

For training, a batch size of 40, an initial learning rate 0.001, and the Adam optimizer were used. The model was trained against the mean absolute error (MAE) between the generated and the target waveform.

Before training, each source signal was normalized to zero mean and unit variance and the target signals were globally normalized across all instances such that all target BP wave-forms lie in the range between −1 and +1. The statistics from training set used for normalization were later applied to the generated waveforms during the testing. The slope K₁ and the y-intercept K₂ in Equation (1) are known to be subject-specific, thus requiring subject-specific calibration for the absolute estimation of BP from pulse transit time. In addition, the ballistocardiogram heartbeat morphology is known to be affected by the postural changes; requiring posture-specific calibration as well.

$$BP = \frac{{K}_{1}}{PTT} + {K}_{2} \qquad ( 1 )$$

To explore the effects of such variability, linear regression was performed independently on subject-specific SBP, DBP, SBP, and inverse pulse transit time pairs to determine a pair of K₁ and K₂ coefficients for each of the three BP components per subject. The calibration coefficients were determined by finding the slope and y-intercept that best fit the calibration data.

For calibration, the first two minutes of recording during the baseline period were used. For testing, the obtained calibration curve was applied to the remaining data points for each subject. To perform linear regression robust to outliers, 5-fold cross-validation was used and the slope and intercept of the model that achieved the highest cross-validation score were determined as K₁ and K₂. For the evaluation, the 10-point moving average was done without overlap to aggregate 10 BP predictions together. Here each prediction was from one ensemble-averaged heartbeat. For the channel selection, the channel with the best signal quality was chosen during the calibration and the same channel was used for the testing as well.

For the U-Net models, the peaks and valleys were detected from the generated BP waveform in each window and served as relative SBP and DBP estimates. These relative SBP and DBP estimates correlated in trend with the true SBP and DBP values, however, the absolute values were often offset by a certain bias and thus required subject-specific re-scaling.

$$bias = \frac{1}{M} {\sum }_{n = 1}^{M} {BP}_{true} ( n ) - {BP}_{est , relative} ( n ) \qquad ( 2 )$$

The relationship between the BP and the pulse transit time is commonly modeled by the MoensKorteweg. To re-scale relative SBP and DBP estimates back to the actual BP range specific to each subject, the bias was measured from the average offset of the first 13 instances that corresponds to 15 seconds of both the true and relative estimations of SBP and DBP as shown in Equation (2). Here, although the offsets were measured from the average of continuous BP over a few seconds, in practice, this could be measured from multiple (e.g. two to three) single-time point BP measurements from a conventional oscillometric cuff without having continuous BP waveform measurements. The relative SBP and DBP estimates were offset by this baseline bias to be re-scaled to the subject-specific BP range. For the evaluation, the mean absolute error (MAE) between the estimated and the reference BP values and the overall Pearson's correlation r were reported. Also, the Bland-Altman plots were presented for the final model to understand the error distribution.

To assess effects of different sensing modalities (toe/finger PPG, and ballistocardiogram), two pulse transit time-based models, the toe pulse transit time (tPTT) and finger pulse transit time (fPTT) and the two U-Net models, a single-modal model using PPG alone and the multi-modal model using both ballistocardiogram and PPG—were evaluated. For the evaluation of the calibration or re-scaling frequency pertinent to postural variability, the calibration or re-scaling was performed in two ways; per day and posture and per day only. In the per day and posture scheme, the calibration was performed per day separately for each posture and subject. In the per day scheme, the posture-specific calibration was removed and the calibration was performed once per day only in supine posture for each subject.

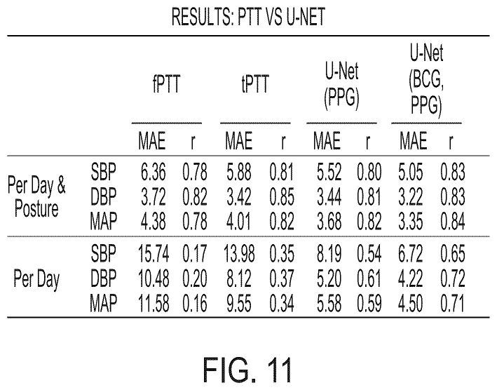

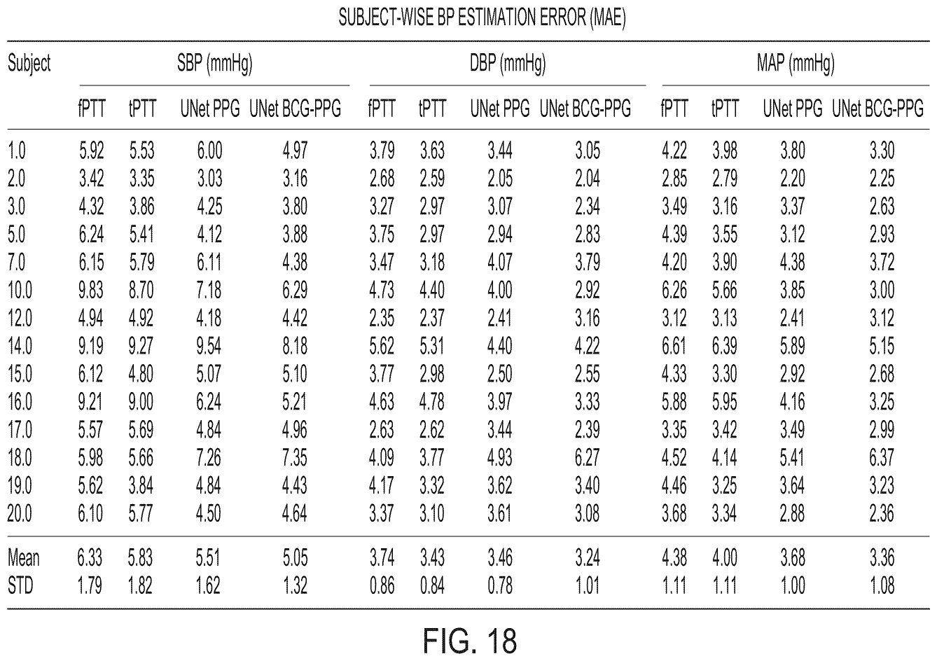

FIG. 11 presents the overall BP estimation error after calibrating the inverse of pulse transit time to the SBP and DBP separately. The finger pulse transit time (fPTT) here indicates the time delay between the J-wave of ballistocardiogram and the maximum derivative of finger PPG and toe pulse transit time (tPTT) indicates the same calculation, but using toe PPG instead. When calibration was performed per day and posture for each subject the overall correlation between the true and the estimated BP was 0.78 and 0.82 for fPTT and 0.81 and 0.85 for tPTT respectively for SBP and DBP. For the MAP, the correlation was similar; 0.78 and 0.82 for fPTT and tPTT, respectively. The mean absolute error (MAE) was lower in tPTT as well as shown in FIG. 18, where the average MAE across subjects were 6.33 mmHg and 3.74 mmHg in fPTT and 5.83 mmHg and 3.43 mmHg in tPTT for SBP and DBP.

The overall BP estimation errors for the U-Net-based methods are shown on the last two columns in FIG. 11. Like the pulse transit time-based methods, two different U-Net modality configurations were evaluated; the single-modality U-Net (i.e. PPG signals only) and the multi-modality U-Net (i.e. ballistocardiogram and PPG signals). When re-scaled per day and posture for each subject, the overall correlations between the true and the estimated values were 0.80, 0.81, and 0.82 in single-modal U-Net and 0.83, 0.83, and 0.84 in multi-modal U-Net for SBP, DBP, and MAP, respectively. In the multi-modal U-Net, this corresponds to a MAE of 5.05 mmHg, 3.22 mmHg, and 3.35 mmHg for SBP, DBP, and MAP respectively as shown in FIG. 18. Therefore, the overall results for the multi-modality model were similar to the fPTT in this calibration scheme with slightly lower MAE compared to the tPTT.

When only the PPG signal was used as a source, the average MAE for SBP, DBP and MAP increased to 5.51 mmHg, 3.46 mmHg, and 3.68 mmHg compared to when both ballistocardiogram and PPG were used as the source. Here the same U-Net architecture and the hyperparameter set were used for the two models but only the source signals were different. Note that with the U-Net-based method, only the finger PPG was used as the public BCG dataset used for the model training only has the finger PPG measurements.

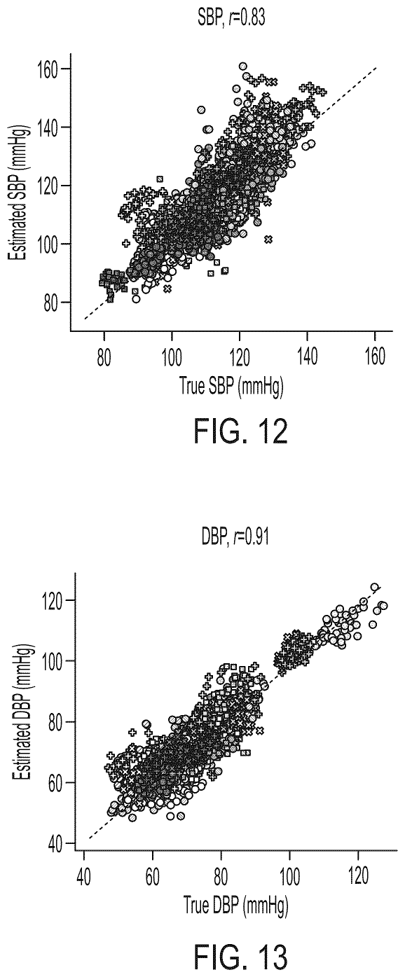

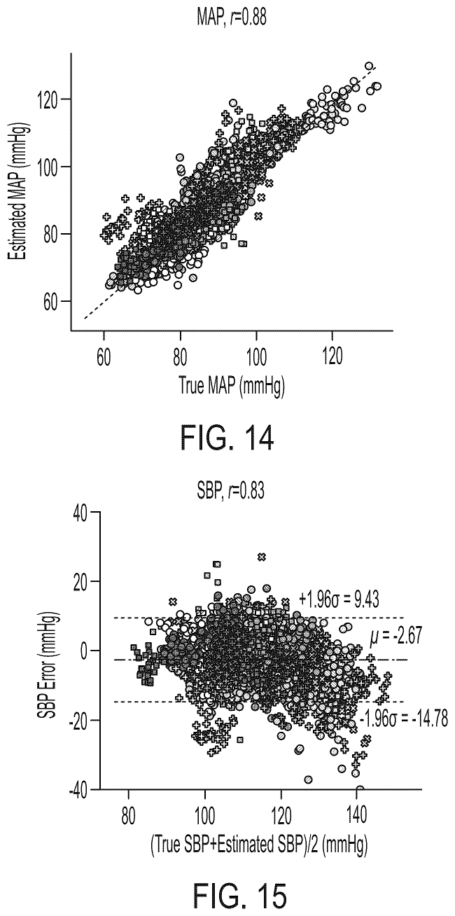

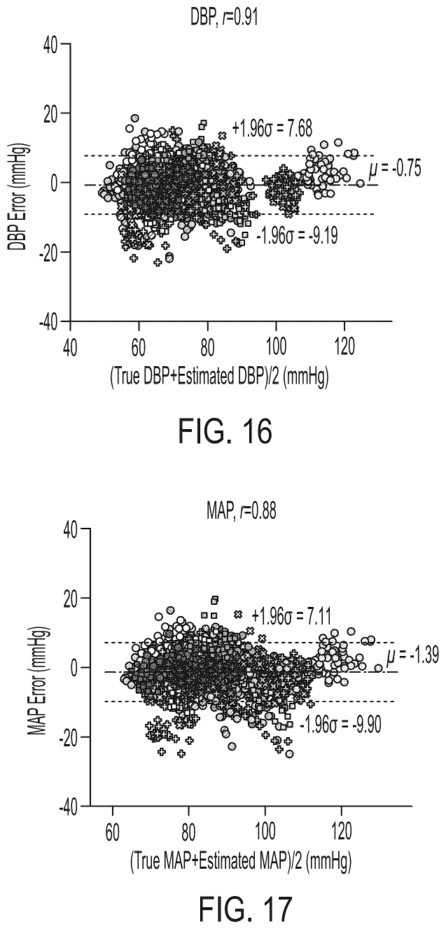

FIG. 12-17 illustrates Bland-Altman plot for the best performing model the multi-modal U-Net. On the plot, the subjects were represented with different colors and the postures were represented with different shapes. FIGS. 12-14 show the overall correlation between the estimated and true values for each BP component. FIGS. 15-17 the error distribution, where the dotted line shows the 95% limit of agreement (LoA). The 95% LoA were [−14.78, 9.43], [−9.19, 7.68], and [−9.90, 7.11] in mmHg for SBP, DBP and MAP. In this section, the posture specific calibration was removed, and the calibration was performed once per day during the first 2 minutes of supine posture for each subject. The calibration curve obtained here was applied to the remaining datapoints from other postures on the same day. FIG. 11 shows the overall results for this calibration scheme.

Without the posture-specific calibration, the correlation has decreased to 0.17, 0.20, and 0.16 in fPTT and 0.35, 0.37, and 0.34 in tPTT for SBP, DBP, and MAP. Similarly, the MAE has increased to 15.74 mmHg, 10.48 mmHg, and 11.58 mmHg in fPTT and 13.98 mmHg, 8.12 mmHg, and 9.55 mmHg in tPTT for SBP, DBP and MAP.

Similarly, for the U-Net-based methods, when re-scaled once per day only, the MAE has increased to 8.19 mmHg, 5.20 mmHg, and 5.58 mmHg in the single-modal model for SBP, DBP, and MAP. The increase in error was observed in the multi-modal model as well; the MAE was 6.72 mmHg, 4.22 mmHg, and 4.50 mmHg with overall correlation of 0.65, 0.72, and 0.71 for SBP, DBP, and MAP. Still, the multi-modal U-Net performed better than the single-modal U-Net or the pulse transit time-based methods. All results here are presented in FIG. 11 and subject-wise MAE is also shown in FIG. 18 for all four models used in the analysis. Note that only the subjects available for both the pulse transit time and U-Net approach were included in the results. When comparing the estimation results between the fPTT and tPTT in FIG. 11 and FIG. 18, the tPTT achieved higher accuracy given the same calibration scheme (per day and posture calibration). Better correlation with toe PPG was also observed in the prior work of R. C. Block, et al., “Conventional pulse transit times as markers of blood pressure changes in humans,” Scientific Reports, vol. 10, no. 1, 2020 (“Block”), which is incorporated by reference herein. In Block, the toe pulse arrival time (the time delay between the ECG R-peak and the foot of the toe PPG waveform) correlated better than the finger pulse arrival time, particularly for the SBP than DBP. Though Block mainly focused on pulse arrival time, it also showed that the pulse transit time detected from the time delay between the ear PPG and toe PPG yielded better correlation compared to the corresponding pulse transit time with finger PPG.

This could be due to smooth muscle contraction which is a well-known confounder of the pulse transit time-based BP monitoring. PPG sensors are most commonly placed at the finger as well as other upper extremity locations to obtain a distal timing reference for pulse transit time extraction. However, these locations are often confounded by the smooth muscle contraction which leads to decrease in correlation to BP. Lower extremity locations such as the toe are less susceptible in that sense as it enables pulse transit time to be measured through the aorta. In addition, the toe is less susceptible to motion artifacts as most people tend to move their fingers more than their toes. However, the finger PPG or the other upper extremity locations such as the wrist are mostly preferred as they are more convenient to measure with non-invasive devices such as smartwatches and finger pulse oximeters. With the advances in deep learning technologies, several studies have demonstrated using the PPG signal alone to generate BP waveform using the state-of-the-art deep architectures. As the PPG waveform by nature has morphological similarity to the BP waveform, these studies have shown success on some levels. However, using the PPG signals alone to estimate BP poses some limitations. Due to the viscoelasticity of the wall of small peripheral arteries for which PPG is applied, the PPG waveform tends to be smoother in morphology and delayed in phase relative to the BP waveform despite its morphological similarity. Also, the PPG sensor contact pressure is an additional complication factor to the PPG waveform. Therefore, relying only on PPG waveforms to monitor BP could lead to some errors.

Conventionally, in many physiology-based analyses pulse transit time using ballistocardiogram and PPG as a proximal and distal reference has shown a better correlation to BP than the PPG morphological features (i.e. peak amplitudes) or the pulse arrival time (the time delay between ECG R-peak and foot of the PPG). However, relatively less studies have been done in deploying DL models with multiple sensing modalities such as a combination of PPG and ballistocardiogram as very few datasets with simultaneously measured PPG, ballistocardiogram, and continuous BP are available to train and evaluate such DL models.

Therefore, both the PPG and ballistocardiogram signals were evaluated for generating BP waveforms using the DL models. Using both ballistocardiogram and PPG signals leverages the morphological similarity of PPG waveforms to BP waveforms while allowing the model to learn the underlying relationship between PPG and ballistocardiogram signals which better constructs the target BP waveform.

The results in FIG. 11 support this idea by showing the improved performance in the multi-modal U-Net model. This suggests the feasibility that the DL model could learn both the morphological and inter-modality relationship to generate a more realistic BP waveform. Despite a larger correlation between the toe PPG and the BP as discussed in the previous section, for the U-Net model, the finger PPG was used over toe PPG as only the finger PPG was available for both the public BCG dataset and the in-house dataset. In future studies, the multi-modal U-Net model with the toe PPG could be investigated to further improve BP waveform generation. From the results in FIG. 18, the tPTT model and multi-modal U-Net performed comparably with slightly lower MAE with the U-Net model when calibrated per day and posture for each subject. Without posture-specific calibration in per day calibration scheme, though the correlation has decreased for both models, the U-Net model yielded significantly better correlation compared to the tPTT model. This demonstrates that while pulse transit time is heavily affected by the posture, the U-Net model is relatively robust to postural variability.

One reason behind the significant drop in estimation accuracy in the pulse transit time-based model could be morphological distortion in the ballistocardiogram caused by the posture. The postural effect on ballistocardiogram morphology is a well-known challenge in ballistocardiogram monitoring, particularly for the bed-based system. Such distortion leads to changes in detected pulse transit time and thus varies the linear mapping between the pulse transit time and the BP according to the postural changes. This requires posture-specific calibration to track absolute BP values. Also, for the pulse transit time, although some noisy beats are removed through the signal quality indexing, the fiducial point detection could be challenging in some postures like the laterals, leading to error in pulse transit time extraction and ultimately inaccurate BP predictions. Rather than having a fixed threshold for removing the beats, dynamic thresholding on SQI further improves pulse transit time extraction.

The channel was chosen based on signal quality assessment during the calibration period (during the baseline period in supine in the per day calibration scheme) to predict the BP values during the test period as different channels often have different calibration curves. However, the best channel during the calibration may not necessarily be the best during testing. The optimization of channel selection or a combination could be done to improve in estimation accuracy in the pulse transit time-based model. To estimate the absolute BP values for the pulse transit time and U-Net models, the extracted pulse transit time or the BP waveform is calibrated for each subject.

For pulse transit time calibration, the K₁ and K₂ coefficients in Equation (1) are known to be person-specific. The K₁ value is determined by the underlying baseline vascular stiffness, whereas K₂ represents the inherently correlated bias in baseline BP; which all varies from person-to-person.

Also, particularly for the bed-based ballistocardiogram system like the one the morphological distortion in ballistocardiogram is often observed, leading to changes in fiducial point timings in the heartbeat. In addition, physiologically, the BP itself is known to be affected by the posture as well. Having both the morphological distortion in ballistocardiogram and the postural effects on BP, it is not surprising that the calibration curve is required per posture and posture for accurate BP estimates.

Therefore, when removing the posture-specific calibration, the estimation accuracy decreased for both pulse transit time and U-Net methods. However, compared to the pulse transit time-based model, the U-Net model's performance was less affected by the postural changes and the DBP error remained under grade A criteria for wearable BP monitoring devices based on the IEEE standard.

Though the U-Net model performed better than the pulse transit time-based model overall, the generated BP waveform from the U-Net model could not be used directly without subject-specific re-scaling to estimate absolute BP values unlike some studies using the DL model. In D.-K. Kim, et al., “DeepCNAP: A Deep Learning Approach for Continuous Noninvasive Arterial Blood Pressure Monitoring Using Photoplethysmography,” IEEE Journal of Biomedical and Health Informatics, p. 1, 2022. (“Kim”) which is incorporated by reference herein, for example, the globalized DL model generated BP waveforms from single-channel PPG signals with high estimation accuracy (MAE of 3.50 mmHg for SBP, 1.81 mmHg for DBP). However, the output from the U-Net model required re-scaling for a new subject to achieve high estimation accuracy. This could be because of the limited number of subjects (n=40) used for the training. Also, using the finger cuff as our target BP waveform may not be as accurate as using the arterial BP in MIMIC II Waveform dataset used in other studies. The BP measured by a finger BP cuff serves as a robust surrogate for ground truth continuous BP measurements and has been validated in previous studies. However, signals from this device could have some measurement errors, particularly while calibrating. Therefore, the measurements from this device may not be suitable as a reference for the BP “waveform” although it could provide robust SBP and DBP values when averaged over a few seconds.

While the dataset used includes a limited number of subjects (20 subjects for the in-house, 40 subjects for the public BCG dataset), all subjects in the analysis were relatively young and healthy without hypertension or any known history of cardiovascular diseases. To mitigate this, the protocol for the human subjects study included perturbations to modulate BP such that large variability in measured BP can be observed even with the limited number of subjects. However, high BP instances were still rare as shown in FIGS. 5-8, thus relatively large error was observed for the subjects with relatively high BP compared to other subjects in the dataset. Those of ordinary skill in the art will recognize that additional subjects with diverse demographics and potentially hypertensive populations will improve the robustness of the model. Also, as observed in previous BP estimation studies, the calibration remains a key challenge. Particularly with the postural changes, more frequent calibration had to be done for both pulse transit time and U-Net models. With more data, future work could further investigate the postural effects on ballistocardiogram and BP signals to reduce calibration requirements. Considering that this bed-based ballistocardiogram system is mainly aimed at the hospital settings where measuring BP with conventional methods such as oscillometric cuff at least once a day is common, reducing the calibration requirement to just once per day could have significant meaning.

The estimation accuracy could be further improved. Though the results were evaluated against the in-house dataset which is more challenging due to surrogate ground truth measurements and extra complications from the postural variability, the estimation error was relatively larger than other studies using the well-studied MIMIC II dataset. For pulse transit time-based models, the utilization of four load cell channels could be optimized to further improve the pulse transit time estimation. For the U-Net models, the variants of the U-Net architecture—adding the attention gates or residual blocks, for example—or other state-of-the-art DL architectures such as LSTM, CNN-LSTM or GAN may also improve the accuracy.

Two different approaches are disclosed for the extraction of the BP features; the conventional pulse transit time-based method and the BP waveform estimation using the DL model. When calibrated per day and posture for each subject, the conventional pulse transit time-based and U-Net-based models performed similarly. In the pulse transit time-based method, the toe pulse transit time correlated better to BP than finger pulse transit time, and in the U-Net-based method, the multi-modal model outperformed a single-modal equivalent. Without the posture-specific calibration, the U-Net model showed a more robust performance compared to the pulse transit time model. Although the estimation error has increased in general with less frequent calibration in both approaches, the estimation accuracy for DBP remained relatively small with U-Net models, showing the feasibility of using the sensors already available in the hospitals, the load cells on the hospital bed and the pulse oximeters, to continuously monitor the BP. Yet, frequent calibration, for each subject per day and posture, is required to achieve high estimation accuracy. Thus, the present disclosure provides a method and apparatus for a bed-based estimation of blood pressure using bed-based sensors for ballistocardiogram and peripheral photoplethysmogram which allows for real-time identification of blood pressure, which can be used as part of a scoring system, such as the early warning score, or modified early warning score, to diagnose the degradation of the health of a patient supported on a hospital bed. Such a diagnosis is critical to early treatment and intervention for any of a number of conditions that may be identified by the early warning score allowing the patient to be receive increased surveillance, additional diagnostic activities, respiratory therapy, cardiac therapy, antibiotic treatments, surgical intervention, or any appropriate therapy that may be identified as necessary due to an early warning score identification of the degradation of the patient. Using the hospital bed systems using force sensors such as the load cell and piezoelectric film have shown robust, continuous measurements of HR and RR in conjunction with the approaches of the present disclosure, the early warning score analysis may be automated to provide feedback to caregivers regarding the patient, while limiting the amount of time that a caregiver has to collect vitals. The addition of the use of a real-time temperature monitor, as is known in the art, can supplement the available data and increase the accuracy of the early warning score, in real-time.

While the disclosure has been illustrated and described in detail in the drawings and the foregoing description, such illustration and description are to be considered illustrative or exemplary and not restrictive. The disclosure is not limited to the disclosed embodiments. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

Although this disclosure refers to specific embodiments, it will be understood by those skilled in the art that various changes in form and detail may be made without departing from the subject matter set forth in the accompanying claims.

1. A method of estimating a blood pressure of a person comprising:
collecting a signal of each of a plurality of load cells supporting the person to determine a ballistocardiogram;
collecting a signal from a photoplethysmogram signal from an appendage of the person; and
applying a model to the signals to determine an estimate of the blood pressure of the person.

2. The method of claim 1, wherein the model includes:
processing the ballistocardiogram and photoplethysmogram signal to filter the signals;
determining a timing delay between the fiducial points in the ballistocardiogram signal and the photoplethysmogram signal to determine a pulse transit time; and
estimating the blood pressure of the person by calculating the inverse of the pulse transit time.

3. The method of claim 1, wherein the model includes:
applying a deep learning model to infer the person's blood pressure.

4. The method of claim 3, wherein the deep learning model includes a contractive path and an expansive path.

5. The method of claim 4, wherein the contractive path reduces the dimension of signal by half while doubling the number of channels for each layer.

6. The method of claim 4, wherein the contractive path includes cascaded layers of convolution, batch normalization, and non-linear activation.

7. The method of claim 6, wherein the expansive path includes de-convolutions.

8. The method of claim 1, wherein the method further comprises calibrating the model for the specific person.

9. The method of claim 8, wherein the calibration is for the absolute estimation of BP from pulse transit time for the specific person.

10. The method of claim 9, wherein the calibration accounts for the specific posture of the person as the model is being applied.

11. The method of claim 8, wherein the calibration accounts for the specific posture of the person as the model is being applied.
